(19) United States
(12) Patent Application Publication
(10) Pub. No.: US 2016/0022749 A1
(43) Pub. Date: Jan. 28, 2016

(54) METHOD OF IMPROVING HEMATOLOGICAL PARAMETERS AND BRAIN DYSFUNCTION WITH GANODERMA TSUGAE EXTRACT

(71) Applicants: Szu-Ming Chen, New Taipei City (TW); Ruey-Shyang Hseu, Taipei (TW)

(72) Inventors: Szu-Ming Chen, New Taipei City (TW); Ruey-Shyang Hseu, Taipei (TW)

(21) Appl. No.: 14/809,637

(22) Filed: Jul. 27, 2015

Related U.S. Application Data
(60) Provisional application No. 62/029,082, filed on Jul. 25, 2014.

(51) Int. Cl. A61K 36/074; A61K 38/44

(52) U.S. Cl. CPC A61K 36/074; A61K 38/446; C12Y 115/01001

(57) ABSTRACT

The invention relates to a method of improving hematological parameters and brain dysfunction by administrating an extract of Ganoderma tsugae. Particularly, the invention relates to a method of using an aqueous or ethanol extract to improve hematological parameters and brain dysfunction.

### FIELD OF THE INVENTION

The invention relates to a method of improving hematological parameters and brain dysfunction by administrating an extract of Ganoderma tsugae. Particularly, the invention relates to a method of using an aqueous or ethanol extract to improve hematological parameters and brain dysfunction.

### BACKGROUND OF THE INVENTION

Hemorheology, also known as haemorheology, or blood rheology, is the study of flow properties of blood and its elements (i.e., blood plasma and cells). Proper tissue perfusion can occur only when blood's rheological properties are within certain levels. Alterations of these properties play significant roles in disease processes such as cardiovascular diseases and brain dysfunction. From a medical standpoint, the importance of studying the viscoelastic properties of blood is evident.

The fungus Ganoderma is a well-known source of antioxidants that has been used in traditional Chinese medicine to treat a variety of nervous system disorders. Extracts of G. lucidum contain antioxidants are absorbed quickly after ingestion, which causes an increase in the total antioxidant activity in human plasma (Sissi Wachtel-Galor, John Yuen, John A. Buswell, and Iris F. F. Benzie, “Herbal Medicine: Biomolecular and Clinical Aspects. 2nd edition-Chapter 9 Ganoderma lucidum (Lingzhi or Reishi),” Benzie IFF, Wachtel-Galor S, editors, 2011). Administering treatment with Extracts of G. lucidum can considerably remedy depression and short-term memory loss (Zhou Y, Qu Z Q, Zeng Y S, Lin Y K, Li Y, Chung P, Wong, R Hagg U, “Neuroprotective effect of preadministration with Ganoderma lucidum spore on rat hippocampus,” Exp Toxicol Pathol, 2011 Jan. 15). Aqueous extracts of G. lucidum beneficially affect neurons by antagonizing Aβ neurotoxicity (Cora Sau-Wan Lai, Man-Shan Yu, Wai-Hung Yuen, Kwok-Fai So, Sze-Yong Zee, “Raymond Chuen-Chung Chang. Antagonizing β-amyloid peptide neurotoxicity of the anti-aging fungus Ganoderma lucidum” BRAINRESEARCH 1190(2008) 215-224), and also exhibit antitumor and immunoregulatory activities. Administering treatment with HEGT decreases the serum level of cholesterol, and improves the healing of acetate-induced ulcers in rats. US 20100278855 provides methods for treatment of degenerative neurological disorders such as Parkinson's disease and Alzheimer's disease and a method for inhibiting the activation of microglial cells by applying the G. lucidum extract obtained by low temperature extraction to the cells comprising administration of a G. lucidum extract.

However, whether extracts of G. tsugae can protect against aluminum-induced learning and memory dysfunction is still unknown.

### BRIEF DESCRIPTION OF THE DRAWINGS

FIG. 1 shows a memory task result for each group and effects of YL1 and YL2 on escape latency (Panel A) and search distance (Panel B) of AlCl₃ induced rats. The rats of AD groups display long escape latency and search distance. (*=P<0.05 vs Control).

FIG. 2 shows effects of YL1 and YL2 on contralateral quadrant (Panel C) and duration time of target quadrant (Panel D). The search time in the target quadrant significantly increases in the groups administered with Ganoderma lucidum fruit extract. (* P<0.05 vs Control).

FIG. 3 shows rCBF analysis of hippocampus and cortex in different fore areas of rat brain. Depending on rCBF result, the blood flow is reduced at the hippocampus (black arrows) and cortex (white arrows) significant in the (+)control group (Panel B), and the flow will rise after using Ganoderma lucidum therapy in YL1 (Panel C), YL2 (Panel D) groups. (Unit: ml/100 g×min), when compared with the control group (Panel A).

FIG. 4 shows the angiography image results of a TOF-MRA scan. In each group, it can be seen that the blood vessel intensity is different in a brain specific area (red circle). In control group (Panel A), very high density of blood vessel signal can be detect from MRI. On the other hand, (+)control group (Panel B) has a lower signal that is hard to detect. And the YL1, YL2 groups have a higher signal than the AD group (Panel C and Panel D).

FIG. 5 shows the result of Immunohistochemical stain on the hippocampus and cortex in each groups. The rats in the (+)control group exhibit significantly more Aβ-containing plaques accumulated in the hippocampus and cortex than the control group. However, the rats in the YL1 and YL2 groups exhibit fewer Aβ-containing plaques accumulated in the hippocampus and cortex than the (+)control group (Panels A and B).

### DETAILED DESCRIPTION OF THE INVENTION

The invention is, at least in part, based on the discovery that extracts from the fruiting bodies of G. tsugae (HEGT) can improve hemorheological parameters, learning and memory. The extracts also can improve brain dysfunction.

As used in the specification and claims, the singular form “a,” “an” and “the” include plural references unless the context clearly dictates otherwise. For example, the term “a cell” includes a plurality of cells, including mixtures thereof.

All numerical designations, e.g., temperature, time, concentration, and molecular weight, including ranges, are approximations. It is to be understood, although not always explicitly stated, that all numerical designations are preceded by the term “about.” It also is to be understood, although not always explicitly stated, that the reagents described herein are merely exemplary and that equivalents of such are well known in the art.

According to the invention, the terms “improvement,” “improving” and the like are used herein to generally mean obtaining a desired pharmacologic, physiologic or cosmetic effect. The effect may be prophylactic in terms of completely or partially preventing a condition, appearance, disease or symptom and/or may be therapeutic in terms of a partial or complete cure for a condition and/or adverse effect attributable to a condition or disease. “Improvement” as used herein covers any improvement of a condition, disease or undesirable appearance in a mammal, particularly a human, and includes: (a) inhibiting the disease, condition or appearance, i.e., causing regression of condition or appearance; (b) relieving the disease, condition or appearance, i.e., causing regression of a condition or appearance.

The term “effective amount,” as used herein, is an amount sufficient to effect beneficial or desired results. For example, a therapeutic amount is one that achieves the desired therapeutic effect. This amount may be the same or different from a prophylatically effective amount, which is an amount necessary to prevent onset of disease or disease symptoms. An effective amount can be administered in one or more administrations, applications or dosages.

In one aspect, the invention provides a method for improving at least one hematological parameter, comprising administering an effective amount of an ethanol or aqueous extract of G. tsugae obtained under high temperature to a subject.

In another aspect, the invention provides a method for improving brain dysfunction, comprising administering an effective amount of an ethanol or aqueous extract of G. tsugae obtained under high temperature to a subject.

Ganoderma tsugae is a flat polypore mushroom of the genus Ganoderma. G. tsugae is non-poisonous but generally considered inedible, due to its solid woody nature; however, extracts made from its fruiting bodies allow medicinal use of the compounds it contains. A hot water extraction can be very effective for extracting the polysaccharides; however, an alcohol or alcohol/glycerin extraction method is more effective for the triterpenoids.

In one embodiment, the extract of G. tsugae is an ethanol extract or aqueous extract obtained under high temperature. Preferably, the G. tsugae is G. tsugae RSH 1109. In a further embodiment, the extract of G. tsugae is an aqueous extract obtained under boiling water. Preferably, the aqueous extract is obtained by boiling a solution with G. tsugae and water in a ratio of 1:10 to 30 for 6 to 15 hours. Preferably, the heating time is 8 to 10 hours. Preferably, the aqueous G. tsugae aqueous extract comprises at least 2.5% (w/w) water-soluble polysaccharide, preferably 2.5% (w/w) to 12% (w/w). The aqueous G. tsugae extract further comprises at least 1.0% (w/w) triterpene, preferably 1.0% (w/w) to 6% (w/w) and at least 2.0×10⁷ U/100 g of superoxide dismutase (SOD), preferably 2.0×10⁷ U/100 g to 15×10⁷ U/100 g. More preferably, the aqueous G. tsugae extract comprises about 3.93% (w/w) water-soluble polysaccharide. Preferably, the aqueous G. tsugae extract further comprises about 1.96% (w/w) triterpene and about 4.31×10⁷ U/100 g of SOD.

In another further embodiment, the extract of G. lucidum is an ethanol extract obtained under high temperature. Preferably, the G. tsugae is G. tsugae RSH 1109. In a further embodiment, the extract of G. tsugae is an ethanol extract obtained by heating a solution with G. tsugae and ethanol in a ratio of 1:10 to 30 at 70° C. to 85° C. for 80 to 120 hours. Preferably, the heating time is about 100 hours. Preferably, the extract is obtained using 95% ethanol. Preferably, the ethanol G. tsugae extract comprises at least 0.10% (w/w) water-soluble polysaccharide, preferably 0.10% (w/w) to 1.0% (w/w). Preferably, the ethanol G. tsugae extract further comprises at least 10% (w/w) triterpene, preferably 10% (w/w) to 35% (w/w), and at least 0.5×10⁸ U/100 g of SOD, preferably 0.5×10⁸ U/100 g to 6×10⁸ U/100 g. More preferably, the ethanol G. tsugae extract comprises about 0.22% (w/w) water-soluble polysaccharide. More preferably, the ethanol G. tsugae extract further comprises about 21.7% (w/w) triterpene and 1.32×10⁸ U/100 g of SOD.

The hematological indexes include viscosity, viscoelasticity, thxixitropy and erythrocyte deformability. The hematological parameter includes, but is not limited to, distribution of RBC volume, viscosity of whole blood, RBC aggregation index, erythrocyte electrophoresis index, erythrocyte rigidity index, internal viscosity of erythrocyte and oxygen transport efficiency.

The brain dysfunction involves cognitive disorders. Cognitive disorders are abnormalities of thinking and memory that are associated with temporary or permanent brain dysfunction. The main symptoms of cognitive disorders include problems with memory, orientation, language, information processing, and the ability to focus and sustain attention on a task. Examples of central nervous system (CNS) disorders or conditions that fall within the scope of cognitive disorders include, age-associated memory impairment, mild cognitive impairment, delirium, dementia, Alzheimer's disease, Parkinson's disease, Huntington's disease, mental retardation, cerebrovascular disease, affective disorders, psychotic disorders, Asperger's disorder, autism, neurotic disorders, attention deficit disorders, oppositional defiant disorder, conduct disorder, subdural hematoma, normal-pressure hydrocephalus, brain tumor, head trauma, and brain trauma (DSM IV). Cognitive disorders may be associated with other conditions. For example, memory impairment can be associated with depression or anxiety, psychosis, Down's syndrome, stroke, traumatic brain injury, Huntington's disease AIDS associated dementia, schizophrenia, and attention deficit disorders.

Cognitive impairment is typically manifested by one or more cognitive deficits. Memory impairment is a cognitive deficit characterized by the inability to learn new information or recall previously learned information. Aphasia is a cognitive deficit characterized by a language and/or speech disturbance. Apraxia is a cognitive deficit characterized by the impaired ability to carry out motor activities despite intact motor function. Agnosia is a cognitive deficit characterized by the failure to recognize or identify objects despite intact sensory functions. Cognitive impairment may also be manifested by a disturbance in executive functioning, i.e., planning, organizing, sequencing, and abstracting.

In certain embodiments, a cognitive disorder is a learning disorder. Such learning disorders are known in the art and include autism, dyslexia, Asperger's syndrome, a neurobiological disorder similar to autism and characterized by serious deficits in social and communication skills; specific learning disability, a disorder in one or more of the basic psychological processes involved in understanding or in using spoken or written language, which may manifest itself in an imperfect ability to listen, think, speak, read, write, spell or to do mathematical calculations; dysgraphia, a disorder that causes difficulty with forming letters or writing within a defined space; dyscalculia, a disorder that causes people to have problems doing arithmetic and grasping mathematical concepts; dyspraxia, a problem with the body's system of motion that interferes with a person's ability to make a controlled or coordinated physical response in a given situation; visual perceptual deficit, difficulty receiving and/or processing accurate information from the sense of sight, although there is nothing wrong with vision; and auditory perceptual deficit, difficulty receiving accurate information through auditory means, even though there is no problem with hearing.

It is preferred that the compound utilized in the present invention is used in therapeutically effective amounts.

The physician will determine the most suitable dosage of the present therapeutic agents, which will vary with the form of administration and the particular compound chosen, and which will further vary according patient under treatment, age of the patient, and type of malady being treated. He will generally wish to initiate treatment with small dosages substantially less than the optimum dose of the compound and increase the dosage by small increments until the optimum effect under the circumstances is reached. The compounds are useful in the same manner as comparable therapeutic agents and the dosage level is of the same order of magnitude as is generally employed with these other therapeutic agents.

In a preferred embodiment, the G. tsugae extract is administered in amounts ranging from about 1 mg to about 100 mg per kilogram of body weight per day. This dosage regimen may be adjusted by the physician to provide the optimum therapeutic response. For example, several divided doses may be administered daily or the dose may be proportionally reduced as indicated by the exigencies of the therapeutic situation. The G. tsugae extract may be administered in a convenient manner, such as by oral, intravenous (where water soluble), intramuscular or subcutaneous routes.

The G. tsugae extract may be orally administered, for example, with an inert diluent or with an assimilable edible carrier, or it may be enclosed in hard or soft shell gelatin capsules, or it may be compressed into tablets, or it may be incorporated directly into the food of the diet. For oral therapeutic administration, the G. tsugae extract may be incorporated with excipients and used in the form of ingestible tablets, buccal tablets, troches, capsules, elixirs, suspensions, syrups, wafers, and the like. The percentage of the compositions and preparations may, of course, be varied and may conveniently be between about 5 to about 80% of the weight of the unit. The amount of G. tsugae extract in such therapeutically useful compositions is such that a suitable dosage will be obtained.

The tablets, troches, pills, capsules and the like may also contain the following: a binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavoring agent such as peppermint, oil of wintergreen, or cherry flavoring. When the dosage unit form is a capsule, it may contain, in addition to materials of the above type, a liquid carrier.

Various other materials may be present as coatings or otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavoring such as cherry or orange flavor. Of course, any material used in preparing any dosage unit form should be pharmaceutically pure and substantially non-toxic in the amounts employed. In addition, G. tsugae extract may be incorporated into sustained-release preparations and formulations. For example, sustained release dosage forms are contemplated wherein the G. tsugae extract is bound to an ion exchange resin which, optionally, can be coated with a diffusion barrier coating to modify the release properties of the resin.

The G. tsugae extract may also be administered parenterally or intraperitoneally. Dispersions can also be prepared in glycerol, liquid polyethylene glycols, and mixtures thereof, and in oils. Under ordinary conditions of storage and use, these preparations contain a preservative to prevent the growth of microorganisms.

The pharmaceutical forms suitable for injectable use include sterile aqueous solutions (where water soluble) or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size, in the case of dispersions, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the G. tsugae extract in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are the use of vacuum drying and freeze-drying techniques on the active ingredient plus any additional desired ingredients from previously sterile-filtered solution(s) thereof.

As used herein, “pharmaceutically acceptable carrier” includes any and all solvents, dispersion media, coatings, antibacterial and antifungal agents, isotonic and absorption delaying agents for pharmaceutical active substances which are well known in the art. Except insofar as any conventional media or agent is incompatible with the active ingredient, its use in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specifics for the novel dosage unit forms of the invention are dictated by and directly dependent on (a) the unique characteristics of the active material and the particular therapeutic effect to be achieved, and (b) the limitations inherent in the art of compounding such as active material for the treatment of disease in living subjects having a diseased condition in which bodily health is impaired as herein disclosed in detail.

Without further elaboration, it is believed that one skilled in the art can utilize the present invention to its fullest extent on the basis of the preceding description. The following examples are, therefore, to be construed as merely illustrative and not a limitation of the scope of the present invention in any way.

### EXAMPLE

### Experimental Animals

Twenty-four male Wistar rats, aged 8 to 12 weeks and weighing 220 to 260 g, were purchased from BioLASCO (Taipei, Taiwan). The rats were reared in a dust-free room at 19° C. to 25° C. with 50% to 70% relative humidity, with food and water available ad libitum. The capture, handling, and tagging procedures were approved by the Animal Care and Use Committee (IACUC) of National Taiwan University (IACUC approval no. 20120304 issued to Shu-Wha Lin, PhD).

### Drugs and Reagents

Analytical-grade AlCl₃ was purchased from Acros Organics (New Taipei City, Taiwan). The G. lucidum were cultivated in Yilan County, Taiwan. The highly concentrated aqueous extract of G. lucidum, YL1, was obtained using a boiling water extraction method. The YL1 contained 3.93% water-soluble polysaccharides, 19.6 mg/g triterpene, and 4.31×10⁷ U/100 g of superoxide dismutase (SOD). The highly concentrated ethanol extract of G. lucidum, YL2, was obtained using an 85% ethanol extraction method, and purified using chromatography. The YL2 contained 0.22% water-soluble polysaccharides, 217.13 mg/g triterpene, and 1.32×10⁸ U/100 g of SOD. The catalase, glutathione (GSH), SOD, and malondialdehyde (MDA) kits were purchased from Cayman Chemical (Ann Arbor, Mich., USA). The mouse interleukin (IL)-6 enzyme-linked immunosorbent assay (ELISA) kit and the TNF-α ELISA kit were purchased from Invitrogen (Carlsbad, Calif., USA). The Aβ40 monoclonal antibody (Immunotech β-amyloid stain) was purchased from Dako (Dako, Denmark). All of the other reagents were reagent grade.

### Experimental Design

The rats were first screened using the Morris water maze test to exclude those that demonstrated extraordinary performance. Twenty-four qualified rats were then randomly divided into the negative control (n=6) and experimental (n=18) groups. The experimental rats were given an AlCl₃ solution (500 mg/kg; i.e., 0.5 mL/100 g), and the negative control rats were given 0.5 mL/100 g of saline on the same schedule. Afterward, the experimental rats were fed an AlCl₃ solution (1600 ppm in distilled water) for up to 5 months. The aluminum-induced brain dysfunction model was established after 5 months of administering the AlCl₃ treatments. The experimental rats were divided into 3 experimental subgroups. The positive control group, the YL1 group, and the YL2 group were treated using saline, YL1, or YL2, respectively, for 2 months. Learning and memory were assessed using the Morris water maze test, and the cerebral blood flow was examined using magnetic resonance imaging (MRI) and angiography. After euthanization, cerebrospinal fluid (CSF) was drawn through the foramen magnum, and blood was collected from the jugular and abdominal veins to measure the hematological, hemorheological, biochemical, and immunological variables. Brain tissue specimens were then collected, and the level of Aβ deposition was evaluated using immunohistochemical (IHC) and hematoxylin staining methods.

### Morris Water Maze Task

The Morris water maze consists of a large circular pool, 180 cm in diameter and 75 cm in height, made of black waterproof canvas and a clear acrylic platform, 20 cm in diameter and 47 cm in height, which is placed inside the pool. The pool was filled to a height of 49 cm with water at approximately 23° C., and the upper surface of the platform was 2.0 cm below the surface of the water. The pool was divided into four quadrants (I, II, III, and IV), with the platform located in the center of the quadrant IV. A video camera was attached to the ceiling, placing the entire pool within the field of view (FOV) of the camera.

The standard procedure of using the Morris water maze has been described in previous studies (19-21). After the experimental treatments (AlCl₃ and saline, YL1, or YL2) were completed, the rats were trained twice a day for 5 days, and once a month thereafter. The escape latency and search distance were calculated to evaluate learning and memory. On Day 1 of the adaptation period, the rats were placed in the water pool and allowed to swim freely for 2 min with no platform that could be used to escape. From Day 2 to Day 4, the water maze reference memory task was performed. The rats were placed in the pool and allowed to swim freely until they found the platform and escaped. This task was repeated 4 consecutive times by gently placing them in one of the 4 quadrants in the following order: II, I, III, and IV.

The escape time and the path crossing the water (search distance) were analyzed with Ethovision XT, Version 8.0, computer software (Noldus, Wageningen, The Netherlands). The maximal escape latency was set at 120 s. When a rat was incapable of finding the platform within 120 s, the rat was guided to the platform, and kept there for 30 s. On Day 5, the water maze probe trial was performed. The rats were placed in the pool and allowed to swim for 120 s with no platform that could be used to escape. The amount of time spent swimming in each quadrant and the search distance were recorded for each rat. Rats with normal memory function lingered in the platform quadrant (quadrant IV).

### Preparation of Blood and Brain Specimens

After the experimental treatments and the learning and memory evaluations were completed, all of the rats were euthanized with sodium pentobarbital. Blood was collected by using venipuncture on the jugular and abdominal veins, and the blood was shunted directly into 2 glass vacuum test tubes, one containing 1.5 mg/mL EDTA as an anticoagulant and another without EDTA. Biochemical, immunological, hematological, and hemorheological analyses of the blood specimens were performed. The brain was removed, and the cerebral cortex and the hippocampus were collected. Specimens of the cerebral cortex and the hippocampus were collected and placed in a 10% neutral formalin fixative buffer at 4° C. The remaining cerebral cortex and hippocampus were placed in a 0.9% NaCl solution at 4° C. The cerebral cortex and the hippocampus were homogenized separately at 4° C., and the homogenates were centrifuged at 10 000×g for 10 min. Biochemical, antioxidant, and neurochemical analyses of the supernatants were then performed.

### Hematological and Hemorheological Measurements Complete Blood Count

A complete blood count (CBC) measures the number of red blood cells, white blood cells, and platelets; the total amount of hemoglobin (Hb); the hematocrit (Hct); the mean corpuscular volume (MCV); the mean corpuscular Hb (MCH) in picograms; and the mean corpuscular Hb concentration (MCHC). The CBC was performed for each rat using an automatic cell counter (Coulter LH750, Beckman-Coulter, Fullerton, Calif., USA).

### Hemorheological Variables

Whole blood viscosity (ηWB), plasma viscosity(ηP), the erythrocyte aggregation index (RAI), and the erythrocyte rigidity index (RI) were measured using a computerized autorotational rheometer (HRD). The measurements of ηWB were performed according to the recommendations of Standardized Hemorheological Methods, as described by Baskurt et al. The sequential ηWB values generated at a high shear rate (120 s⁻¹), a medium shear rate (70 s⁻¹), and a low shear rate (30 s⁻¹) were obtained to compare the experimental and negative control rats using a computer-controlled testing program. The internal viscosity of erythrocytes (Tk) was calculated as Tk=[(η0.4-1)η0.4]/Hct, according to the Dintenfass equation. The ηP was detected at a high shear rate of 120 s⁻¹, and the plasma level of fibrinogen was detected using the thrombin clot method. The oxygen transport efficiency (OTE) of the whole blood was calculated at a fixed shear rate as OTE=Hct/ηWB.

### Biochemistry and Immunological Analysis

The serum levels of glucose, triglyceride, cholesterol, high-density lipoprotein cholesterol (HDL-C), total protein, albumin, AST, ALT, Alk-p, folic acid, and high sensitivity C-Reactive Protein (Hs CRP) and the CSF to serum albumin ratio were determined using a Model P800 automatic biochemical analyzer (Roche Diagnostics, Indianapolis, Ind., USA). The plasma level of homocysteine was determined using a Model 7150 automatic biochemical analyzer (Hitachi, Tokyo, Japan). The CSF levels of IL-6 and TNF-α were determined using ELISAs. The acetylcholinesterase activity in erythrocytes (AchE-RBC) was determined using a colorimetric method.

### Regional Cerebral Blood Flow and Angiography Imaging Analyses

The rats were placed in a stereotaxic holder and a respiration monitor was used. Whole-brain imaging was performed with T2-weighted imaging (T2-WI), arterial spin labeling (ASL) and time-of-flight (TOF) angiography using a 7T BioSpec 70/30 MRI system (Bruker, Ettlingen, Germany). The T2-WIs were acquired using the TurboRARE-T2 technique, with a matrix=384×384, an FOV=2.5×2.5 cm, 21 slices (slice thickness=1 mm), a TE/TR=33/2500 ms, and 4 averages. The ASL was performed using a flow-sensitive alternating inversion-recovery echo planar imaging technique, with a matrix of 128×128, FOV=2.5×2.5 cm, an inversion-recovery time (TIR)=100 to 6000 ms, 60 TIR values, a recovery time=10 000 ms, and a TE/TR=25/18 000 ms. The TOF angiography was performed using a FLASH-3D-TOF sequence, with a matrix=256×256, an FOV=2.5×2.5×3.5 cm, a TE/TR=2.5/15 ms, a 20° flip angle, and an NEX=1. All of the image analyses were performed using ParaVision computer software (Bruker) on an MRI console. The maximal intensity projection was used to display the angiography. The ASL images were analyzed using an ASL perfusion processing program, with a blood T1 value in 7T of 2200 ms. The cerebral blood flow (CBF) was derived from the non-selective and selective T1 maps with CBF=λT1 non-selective/T1 blood (1/T1 selective−1/T1 non-selective), where λ is the blood-brain partition coefficient. The blood-brain partition coefficient was defined as the ratio of the water concentration per gram of brain tissue and per millimeter of blood, which was estimated to be 90 mL/100 g for the rats used in the experiments performed in this study. CBF was expressed in units of mL·min⁻¹·100 g⁻¹.

### Glutathione Peroxidase, SOD, Catalase, and MDA Assays

The supernatant of the hippocampus homogenate was used to measure glutathione peroxidase activity using the cumene hydroperoxide method, and SOD activity was determined using the xanthine oxidase method. In addition, catalase activity was determined using the hydrogen peroxide method, and MDA level was determined using a thiobarbituric acid colorimetric method.

IHC Staining and Analysis of Aβ42 signaling in the hippocampus and cortex

The fixed cerebral cortex and hippocampus specimens were embedded in paraffin, and 4-μm sections were prepared. The sections were collected sequentially and placed into wells immediately. Sections of the hippocampus and cortex were analyzed for the presence of Aβ42 using an IHC staining method. IHC analysis was performed in a BenchMark XT automatic staining machine (Ventana Medical Systems, Tucson, Ariz., USA) using an iVIEW 3,3-diaminobenzidine (DAB) Detection Kit (Ventana Medical Systems). After the brain tissue sections were deparaffinized and hydrated, the slides were treated with an iVIEW inhibitor at 37° C. for 4 min to inactivate endogenous peroxidase activity. The slides were then incubated with a 1:150 dilution of a mouse antiAβ-N-terminal monoclonal antibody (clone NT 3F5; MyBiosource, San Diego, Calif., USA) in blocking solution at 37° C. for 16 min. After rinsing with phosphate-buffered saline (PBS), the slides were treated with the iVIEW biotin-conjugated IgG antibody in blocking solution for 8 min at room temperature. The slides were rinsed with PBS again and then incubated with the iVIEW streptavidin-conjugated horse-radish peroxidase in blocking solution for 8 min at room temperature. The Aβ reactivity was visualized by adding the iVIEW DAB and hydrogen peroxide, and incubating for 8 min at 37° C. The slides were incubated with iVIEW copper for 4 min to enhance signal intensity, and counterstained with hematoxylin (Vector Laboratories, Burlingame, Calif., USA). The slides were photographed using a Nikon Eclipse E600 microscope (Tokyo, Japan) equipped with a CCD camera. Aβ42 accumulation in the hippocampus was also evaluated using a snatch microscope examination.

### Statistical Analysis

The statistical significance of the behavioral and biochemical effects was determined using a one-way analysis of variance (ANOVA), followed by an ANOVA containing Duncan multiple comparison tests. The results are expressed as mean±standard error. All of the data were analyzed using an SPSS, Version 17, statistical software package (IBM, Chicago, Ill., USA). The level of statistical significance was set to P<0.05.

### Example 1

### Physiological, Blood, and CSF Biochemical Analyses

A significant increase in body weight was observed after 5 months of administering the AlCl₃ treatment, and an additional increase in body weight was observed after 2 months of administering the HEGT treatments. No significant difference in water or food intake was observed between the experimental groups and the negative control group. The blood and CSF biochemical analyses revealed that the CSF and serum albumin and the concentrations of ALT, fibrinogen, HsCRP, IL-6, and TNF-α were significantly higher in the positive control group, compared with those of the negative control group (Table 1). The concentrations of HsCRP and AchE-RBC in the YL1 and YL2 groups were significantly lower than those of the positive control group. The CSF and serum albumin and the levels of fibrinogen, HsCRP, IL-6, and TNF-α were significantly lower in the YL1 and YL2 groups, compared with those of the positive control group.

TABLE 1
The results of blood biochemistry analysis of AD rats which were induced by aluminum chloride for
five months and then treated with YL1 and YL2 for 2 months. Values are represented as mean ± S.E.M.

| | Control(n = 6) | (+) Control(n = 6) | YL 1(n = 6) | YL 2(n = 6) |
|---|---|---|---|---|
| Glucose(mg/dl) | 115.2 ± 13.4 | 112.2 ± 11.9 | 112.3 ± 15.4 | 111.5 ± 10.9 |
| Triglyceride (mg/dl) | 70.0 ± 15.6 | 73.5 ± 10.0 | 73.7 ± 11.2 | 71.2 ± 11.0 |
| Cholesterol (mg/dl) | 85.8 ± 22.5 | 84.7 ± 8.5 | 83.0 ± 11.7 | 83.7 ± 10.5 |
| HDL-C(mg/dl) | 58.0 ± 7.5 | 55.1 ± 6.5 | 57.7 ± 7.5* | 59.1 ± 6.7 |
| Total protein(g/dL) | 6.2 ± 0.3 | 6.1 ± 0.2 | 6.4 ± 0.6 | 6.2 ± 0.2 |
| Albumin(g/dL) | 3.9 ± 0.5 | 3.9 ± 0.2 | 4.0 ± 0.4 | 3.9 ± 0.2 |
| AST(U/L) | 127.5 ± 29.1 | 130.5 ± 19.2 | 128.8 ± 19.5 | 129.7 ± 17.9 |
| ALT(U/L) | 45.8 ± 8.3 | 47.7 ± 6.7 | 46.2 ± 7.9 | 49.2 ± 9.1 |
| Alk-p | 49.7 ± 7.2 | 51.3 ± 9.5 | 50.8 ± 9.9 | 48.8 ± 5.8 |
| Fibrinogen(mg/dl) | 182.2 ± 6.8 | 200.9 ± 5.4# | 190.3 ± 5.3#* | 183.6 ± 4.0** |
| Homocysteine(μmol/L) | 20.1 ± 1.9 | 22.0 ± 1.1 | 20.4 ± 1.7** | 21.8 ± 2.1 |
| Folic acid(ng/ml) | 24.1 ± 2.0(>23) | 23.2 ± 2.2(>23) | 23.5 ± 2.2(>23) | 24.2 ± 2.5(>23) |
| Hs CRP(ng/dl) | 30.833 ± 6.047 | 58.667 ± 5.820# | 39.833 ± 4.833#** | 42.500 ± 2.881#** |
| IL-6 (CSF) (pg/ml) | 2.194 ± 0.391 | 3.800 ± 0.638# | 2.931 ± 0.686* | 3.210 ± 0.675# |
| TNF-α(CSF) (pg/ml) | 0.512 ± 0.187 | 0.935 ± 0.193# | 0.796 ± 0.180#** | 0.707 ± 0.247 |
| CSF/serum Albumin | N/A | 0.047 ± 0.013# | 0.019 ± 0.006#** | 0.021 ± 0.007#* |
| AChE-RBC | 2.365 ± 0.229 | 4.975 ± 0.462# | 3.120 ± 0.483#** | 3.563 ± 0.466#** |
| (mole/min · L) | | | | |

Values are represented as mean ± SD
*P < 0.05 vs (+) Control,
**p < 0.01 vs (+) Control,
#p < 0.05 vs Control

### Example 2

### HEGT Treatment Improves Learning and Memory in AlCl₃-Treated Rats

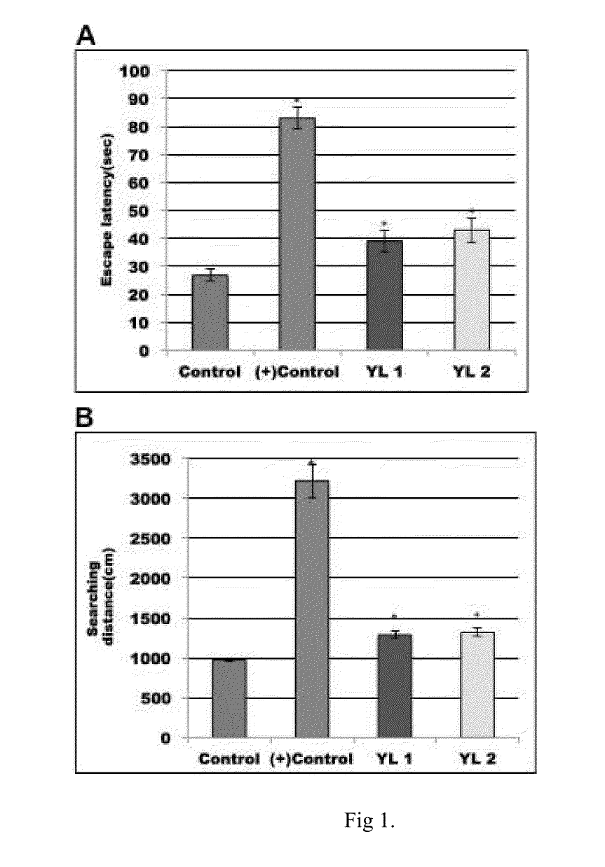

The analysis results regarding the effects of HEGT treatments on memory and learning are shown in FIG. 1. The rats treated with AlCl₃ demonstrated a longer escape latency and search distance than the negative control group did. However, both escape latency and the search distance were significantly lower in the YL1 and YL2 groups compared with those of the positive control group (P<0.05). In addition, we determined that the swimming speed in the YL1 and YL2 groups was faster than that of the positive control group. The 120-s spatial probe trial was followed by the reference memory trial.

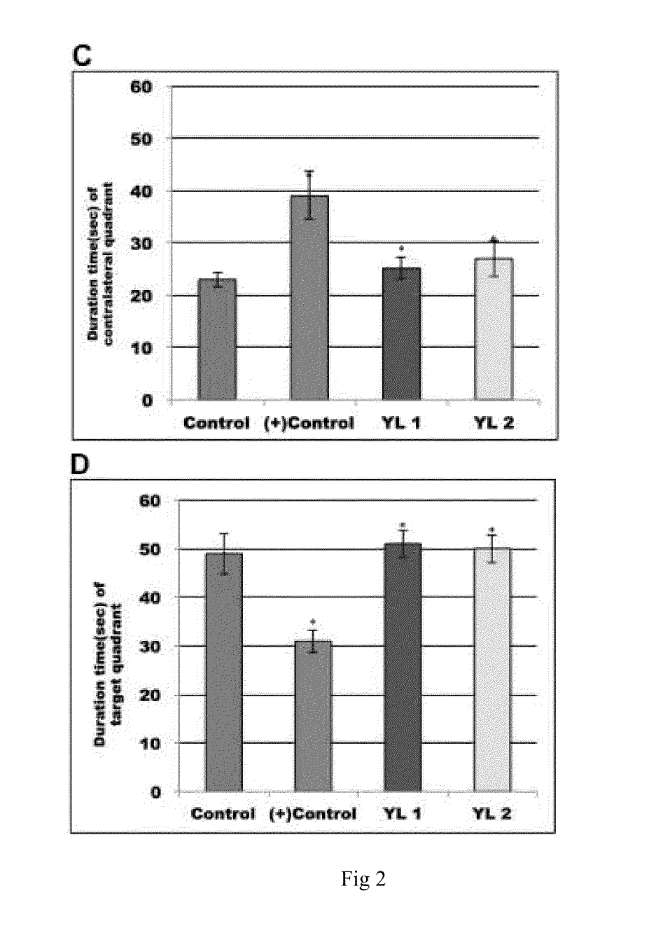

The one-way ANOVA revealed significant differences between the average time that each group spent in the target quadrant and the average time each group spent in the contralateral quadrant (FIG. 2). The positive control group spent less time in target quadrant than the negative control group did (P<0.05). The search time in the target quadrant was significantly higher in the YL1 and YL2 groups. Analyzing the swimming pathway was helpful for evaluating the learning and memory of the rats in the spatial probe trial. The results demonstrated that the positive control rats searched for the target quadrant in a directionless manner by swimming around the entire pool. By contrast, rats in the YL1, YL2, and negative control groups demonstrated a higher capacity for learning and memory by swimming directly to the target quadrant and remaining there for a longer period than the positive control rats did.

### Example 3

### HEGT Treatment Enhanced the Antioxidant Capacity of AlCl₃-Treated Rats

The activities of glutathione peroxidase, SOD, and catalase were reduced in the supernatants of the cerebral cortex and the hippocampus of the positive control rats (P<0.05; Table 2). MDA level was highest in the erythrocytes in the jugular vein blood and the supernatants of the cerebral cortex and the hippocampus of the positive control rats (P<0.05).

TABLE 2
The effects of Ganoderma lucidum fruit Extract administration on activities of Glutathione peroxidase,
Superoxide dismutase, Catalase, and MDA levels of cortex, hippocampus and jugular blood RBC.

| | Control(n = 6) | (+)Control(n = 6) | YL 1(n = 6) | YL 2(n = 6) |
|---|---|---|---|---|
| SOD activity in hippo. | 18.18 ± 0.95 | 10.273 ± 0.84# | 16.02 ± 1.11#** | 14.61 ± 0.59#** |
| (Inhibition rate/mg) | | | | |
| SOD activity in cortex | 2.88 ± 0.16 | 1.30 ± 0.18# | 2.32 ± 0.32#* | 2.02 ± 0.16#** |
| (Inhibition rate/mg) | | | | |
| Catalase Activity in hippo. (mU/ml/mg) | 0.18 ± 0.01 | 0.09 ± 0.01# | 0.15 ± 0.01#** | 0.14 ± 0.59#** |
| Catalase Activity in cortex (mU/ml/mg) | 0.06 ± 0.01 | 0.03 ± 0.01# | 0.05 ± 0.01** | 0.05 ± 0.01#** |
| GPx activity in hippo (mU/ml/mg) | 14.62 ± 1.1 | 8.04 ± 0.73# | 12.01 ± 0.81#** | 10.87 ± 0.63#** |
| GPx activity in cortex (mU/ml/mg) | 1.19 ± 0.02 | 0.70 ± 0.01# | 1.01 ± 0.07#** | 0.95 ± 0.06#** |
| MDA in hippo (nmol/mg) | 0.03 ± 0.01 | 0.12 ± 0.01# | 0.06 ± 0.01#** | 0.07 ± 0.01#** |
| MDA in cortex (nmol/mg) | 0.05 ± 0.01 | 0.13 ± 0.01# | 0.08 ± 0.01#** | 0.09 ± 0.01#** |
| MDA in RBCs of jugular blood | 1.05 ± 0.07 | 3.85 ± 0.18# | 2.07 ± 0.19#** | 2.21 ± 0.18#** |
| (nmol/mg) | | | | |

Values are represented as mean ± SD
*P < 0.05 vs (+) Control,
**p < 0.01 vs (+) Control,
#p < 0.05 vs Control

### Example 4

### HEGT Promoted Regional Cerebral Blood Flow in AlCl₃-Treated Rats

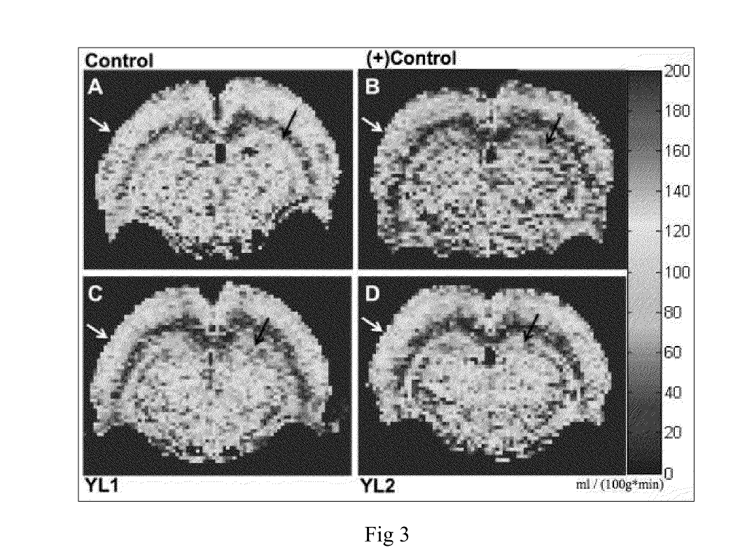

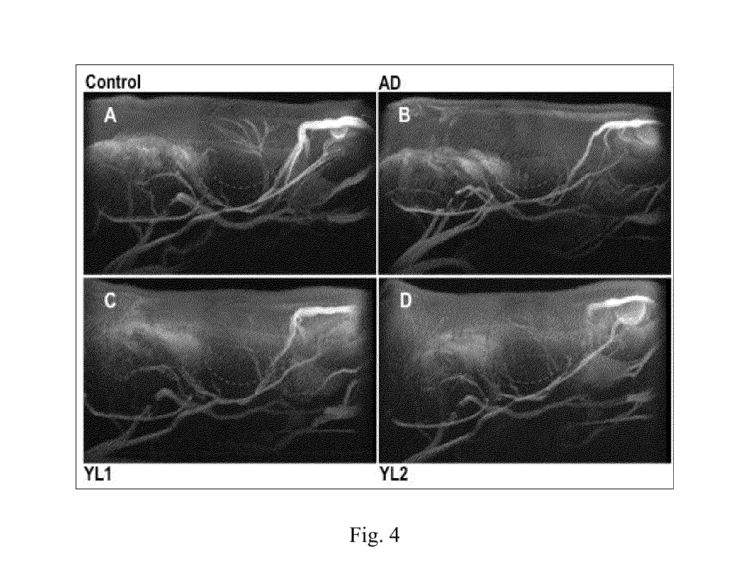

FIG. 3 and Table 3 show the data from the analysis of regional cerebral blood flow (rCBF) and the brain vasculature. The rCBF was reduced by 30% to 40% in the positive control rats compared with that of the negative control rats. However, the rCBF of the rats treated using YL1 or YL2 was approximately 80% of that of the negative control rats. The vascular signal of the positive control rats was lower than the detection limit of using the TOF technique. The vascular signal exhibited in the MRI of the negative control rats was high density, whereas the positive control group exhibited a lower density. TOF imaging revealed that the vessel density around the hippocampus and the cerebral cortex was higher in the rats treated with YL1 or YL2 compared with that of the positive control rats (FIG. 4).

TABLE 3
Comparison of rCBF map in control, (+)control, YL1 and YL2 between hippocampus
and cortex. The quantitative analysis of rCBF including four different regions.

| | Control(n = 6) | (+)Control(n = 6) | YL 1(n = 6) | YL 2(n = 6) |
|---|---|---|---|---|
| Left Cortex | 104.83 ± 14.59 | 76.62 ± 3.73# | 97.90 ± 6.99** | 98.61 ± 16.85** |
| Right Cortex | 107.59 ± 14.54 | 67.01 ± 3.14# | 96.19 ± 7.13** | 101.24 ± 19.20** |
| Left Hippocampus | 92.35 ± 9.49 | 61.47 ± 4.48# | 71.93 ± 5.80#** | 76.30 ± 13.73#** |
| Right Hippocampus | 89.50 ± 8.34 | 62.90 ± 4.55# | 72.65 ± 5.12#** | 75.42 ± 16.55#* |

Values are represented as mean ± SD
*P < 0.05 vs (+) Control,
**p < 0.01 vs (+) Control,
#p < 0.05 vs Control

### Example 5

### HEGT Alters the Hemorheological Parameters of A;Cl₃-Treated Rats

The data used for the comparison of the CBC parameters are shown in Tables 4 and 4′. The results reveal the red blood cell distribution widths (RDWs) of the positive control rats were positive. No significant differences in the CBC parameters were observed between the negative control group and the experimental groups.

Comparison of the hemorheological parameters derived from the jugular vein blood and abdominal vein blood is shown in Tables 5 and 5′. All of the hemorheological parameters examined, including TK, were significantly different between the negative control and positive control groups. The ηWB, the ηP, and the RAI of the positive control rats were significantly higher than those of the negative control rats, whereas the erythrocyte electrophoresis (EI), the erythrocyte deformability index (DI), and the OTE of whole blood were significantly lower in the positive control group than in the negative control group. Furthermore, the ηWB at a low shear rate was significantly higher for the jugular vein blood than for the abdominal vein blood in the positive control group.

The ηWB, the ηP, and the RAI of both the jugular and abdominal vein blood were significantly lower in the YL1 and YL2 groups compared with those in the positive control group (Table 6), and the EI, DI, and OTE of both the jugular and abdominal vein blood were significantly higher in the YL1 and YL2 groups compared with those in the positive control group. However, more Aβ plaques accumulated in the hippocampus and the cerebral cortex of the positive control group than in those of the negative control group, which might have been related to the higher ηWB observed in the positive control rats.

TABLE 4
Hematological parameters from the jugular blood of rats in the
control and (+)control groups show no significant differences.

| | Control(n = 6) | (+)Control(n = 6) | YL 1(n = 6) | YL 2(n = 6) |
|---|---|---|---|---|
| WBC(×10³/mm³⁾ | 2.4 ± 0.4 | 2.2 ± 0.4 | 2.3 ± 0.3 | 2.5 ± 0.5 |
| RBC(×10⁶/mm³⁾ | 8.7 ± 0.2 | 8.6 ± 0.3 | 8.6 ± 0.8 | 8.9 ± 0.6 |
| Hgb(g/dl) | 15.5 ± 0.4 | 16.1 ± 0.4 | 15.7 ± 1.3 | 16.3 ± 0.3 |
| Hct(%) | 45.2 ± 1.7 | 46.0 ± 0.9 | 44.9 ± 3.5 | 46.6 ± 2.1 |
| MCV | 52.9 ± 0.8 | 52.8 ± 0.5 | 52.1 ± 1.3 | 52.2 ± 1.1 |
| MCH | 18.0 ± 0.6 | 18.5 ± 0.2 | 18.3 ± 1.0 | 18.5 ± 0.7 |
| MCHC | 35.4 ± 0.5 | 35.2 ± 0.4 | 35.3 ± 1.1 | 35.5 ± 0.4 |
| Platelet(×10³/μl) | 972.0 ± 55.2 | 1001.0 ± 57.1 | 957.2 ± 82.4 | 959.5 ± 86.8 |

RDW represents the distribution of the RBC volume, high RDW indicating obvious variation in RBC volume.
Data are represented as mean ± SD. (hematology)
*P < 0.05 vs (+) Control,
**p < 0.01 vs (+) Control,
#p < 0.05 vs Control

TABLE 4′
Hematological parameters from the abdominal vein blood of rats in
the control and (+)control groups show no significant differences.

| | Control(n = 6) | (+)Control(n = 6) | YL 1(n = 6) | YL 2(n = 6) |
|---|---|---|---|---|
| WBC(×10³/mm³⁾ | 2.1 ± 0.7 | 2.1 ± 0.2 | 2.1 ± 0.6 | 2.1 ± 0.3 |
| RBC(×10⁶/mm³⁾ | 8.2 ± 0.7 | 8.2 ± 0.1 | 8.5 ± 0.9 | 8.5 ± 0.6 |
| Hgb(g/dl) | 14.7 ± 1.2 | 15.3 ± 0.3 | 15.4 ± 1.5 | 15.4 ± 0.7 |
| Hct(%) | 42.9 ± 4.0 | 43.8 ± 0.6 | 44.1 ± 4.6 | 43.9 ± 2.2 |
| MCV | 51.7 ± 1.2 | 51.9 ± 0.7 | 51.7 ± 1.2 | 51.5 ± 1.2 |
| MCH | 17.9 ± 0.3 | 18.1 ± 0.5 | 18.3 ± 1.0 | 18.0 ± 0.7 |
| MCHC | 34.7 ± 1.0 | 35.2 ± 0.7 | 34.9 ± 1.4 | 35.0 ± 0.4 |
| Platelet(×10³/μl) | 996.3 ± 51.1 | 968.7 ± 55.3 | 930.0 ± 74.5 | 991.0 ± 65.6 |

RDW represents the distribution of the RBC volume, high RDW indicating obvious variation in RBC volume.
Data are represented as mean ± SD. (hematology [symbol] )
*P < 0.05 vs (+) Control,
**p < 0.01 vs (+) Control,
#p < 0.05 vs Control

TABLE 5
Hematological parameters from the jugular blood
of rats in the control and (+)control groups.

| | Control(n = 6) | (+)Control(n = 6) | YL 1(n = 6) | YL 2(n = 6) |
|---|---|---|---|---|
| ηWB(cp) | | | | |
| r = 120 S⁻ | 6.88 ± 0.23 | 9.67 ± 0.48# | 7.80 ± 1.91* | 7.72 ± 1.44* |
| r = 70 S⁻ | 8.19 ± 0.3 | 11.00 ± 0.66# | 9.48 ± 2.05 | 9.16 ± 1.64* |
| r = 30 S⁻ | 11.48 ± 0.46 | 15.83 ± 0.78# | 13.10 ± 3.05 | 13.08 ± 2.34* |
| ηrdWB(cp) | | | | |
| r = 120 S⁻ | 6.66 ± 0.43 | 8.43 ± 0.50# | 6.58 ± 1.19* | 6.28 ± 0.68** |
| r = 70 S⁻ | 8.38 ± 0.44 | 9.91 ± 0.57# | 8.50 ± 1.14* | 7.85 ± 0.65** |
| r = 30 S⁻ | 12.56 ± 0.5 | 14.72 ± 0.64# | 12.60 ± 1.73* | 12.14 ± 0.79** |
| ηP | 1.83 ± 0.09 | 2.22 ± 0.15# | 1.97 ± 0.22 | 1.88 ± 0.18* |
| AI | 4.79 ± 0.55 | 6.11 ± 0.61# | 4.91 ± 0.37* | 4.81 ± 0.23** |
| RI | 6.05 ± 0.62 | 7.47 ± 1.00# | 6.14 ± 0.77* | 6.63 ± 1.05 |
| EI | 13.59 ± 1.36 | 16.15 ± 1.40# | 13.23 ± 1.91* | 13.50 ± 1.82* |
| TK | 0.90 ± 0.05 | 0.93 ± 0.03 | 0.91 ± 0.07 | 0.93 ± 0.03 |
| OTE | 0.07 ± 0.00 | 0.05 ± 0.00# | 0.06 ± 0.01* | 0.06 ± 0.01* |

ηWB: Viscosity of whole blood
cp = mpa · s
ηrdWB: Reduced viscosity of whole blood
RAI: RBC aggregation index
EI: Erythrocyte electrophoresis index
RI: Erythroctye rigidity index
TK: Internal viscosity of erythrocyte
OTE: Oxygen trensport efficiency (or oxygen delivery index) of whole blood = Hct/ηWB
Data are represented as mean ± SD.
*P < 0.05 vs (+) Control,
**p < 0.01 vs (+) Control,
#p < 0.05 vs Control

TABLE 5′
Hematological parameters from the abdominal vein blood
of rats in the control and (+)control groups.

| | Control(n = 6) | (+)Control(n = 6) | YL 1(n = 6) | YL 2(n = 6) |
|---|---|---|---|---|
| ηWB(cp) | | | | |
| r = 120 S⁻ | 6.23 ± 0.37 | 8.32 ± 0.60# | 6.35 ± 0.76** | 6.01 ± 0.95** |
| r = 70 S⁻ | 7.18 ± 0.48 | 9.47 ± 0.65# | 7.54 ± 0.81** | 7.03 ± 0.96** |
| r = 30 S⁻ | 9.84 ± 0.63 | 13.62 ± 0.43# | 11.35 ± 1.12#** | 10.51 ± 0.85** |
| ηrdWB(cp) | | | | |
| r = 120 S⁻ | 5.81 ± 0.61 | 7.44 ± 0.43# | 5.24 ± 1.08** | 4.81 ± 1.20** |
| r = 70 S⁻ | 7.04 ± 0.65 | 8.81 ± 0.56# | 6.66 ± 1.21** | 6.01 ± 1.22** |
| r = 30 S⁻ | 10.54 ± 1.08 | 13.47 ± 1.12# | 11.14 ± 1.24* | 9.93 ± 0.82** |
| ηP | 1.79 ± 0.15 | 2.12 ± 0.13# | 1.88 ± 0.18** | 1.91 ± 0.14* |
| AI | 4.70 ± 0.55 | 5.74 ± 0.85# | 4.73 ± 0.48** | 4.74 ± 0.41* |
| RI | 5.81 ± 0.61 | 7.02 ± 0.74# | 5.71 ± 0.73** | 5.34 ± 0.56** |
| EI | 13.45 ± 0.71 | 15.44 ± 1.15# | 13.19 ± 1.18** | 13.48 ± 1.47** |
| TK | 0.92 ± 0.07 | 1.00 ± 0.05 | 0.88 ± 0.09** | 0.86 ± 0.07** |
| OTE | 0.07 ± 0.01 | 0.05 ± 0.00# | 0.07 ± 0.00** | 0.07 ± 0.01** |

ηWB: Viscosity of whole blood
cp = mpa · s
ηrdWB: Reduced viscosity of whole blood
RAI: RBC aggregation index
EI: Erythrocyte electrophoresis index
RI: Erythroctye rigidity index
TK: Internal viscosity of erythrocyte
OTE: Oxygen trensport efficiency (or oxygen delivery index) of whole blood = Hct/ηWB
Data are represented as mean ± SD.
^{≠} No significant difference vs. the (+)control group (hemorheology)
*P < 0.05 vs (+) Control,
**p < 0.01 vs (+) Control,
#p < 0.05 vs Control

### Example 6

### HEGT Treatment Reduces Aβ Deposition in the Hippocampus and the Cerebral Cortex

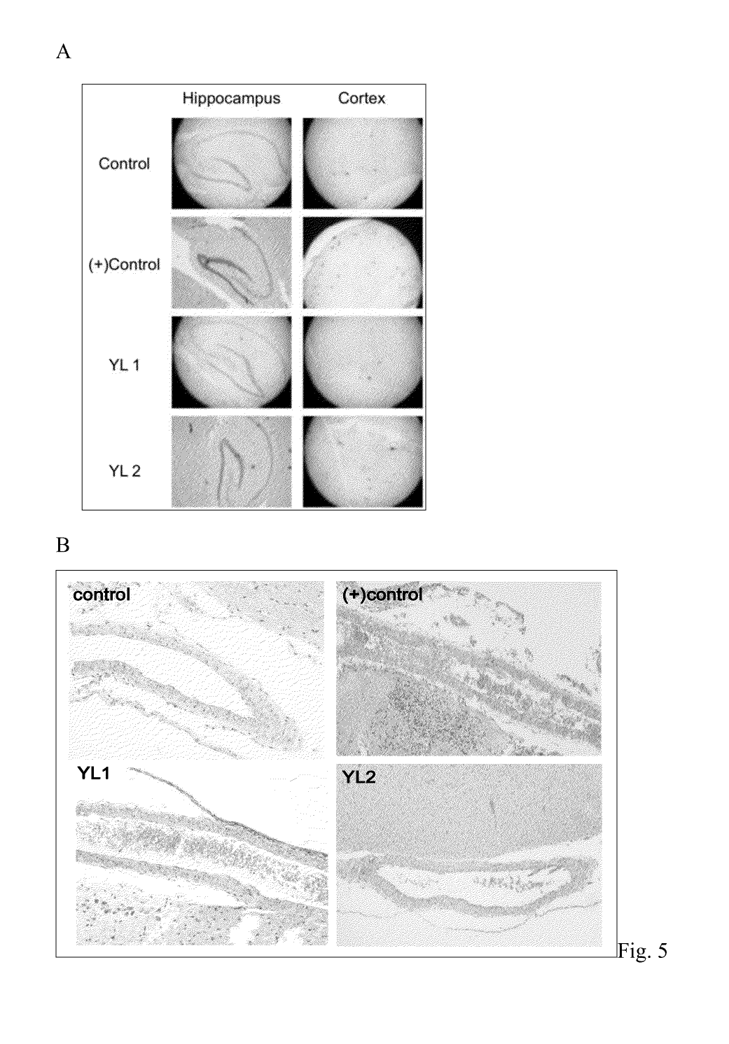

The effect of treatment with YL1 or YL2 on the numbers of Aβ plaques in the hippocampus and the cerebral cortex of AlCl₃-treated rats was evaluated using IHC and hematoxylin staining. The Aβ40 plaques were visible as dark-red colored areas in the hippocampus and the cerebral cortex sections (FIG. 5), and the microscopic examination (×100 magnification) indicated that significantly more Aβ plaques accumulated in the brains of the positive control rats, compared with those in the negative control, YL1, or YL2 groups.

What is claimed is:

1. A method for improving at least one hematological parameter or a brain dysfunction, comprising administering an effective amount of an ethanol or aqueous extract of G. tsugae obtained under high temperature to a subject.

2. The method of claim 1, wherein the G. tsugae is G. tsugae RSH 1109.

3. The method of claim 1, wherein the aqueous extract of G. tsugae is obtained by boiling a solution comprising G. tsugae and water in a ratio of 1:10 to 30 for 6 to 15 hours.

4. The method of claim 3, wherein the heating time is 8 to 10 hours.

5. The method of claim 1, wherein the aqueous G. tsugae aqueous extract comprises at least 2.5% (w/w) water-soluble polysaccharide.

6. The method of claim 5, wherein the aqueous G. tsugae extract further comprises at least 1.0% (w/w) triterpene and at least 2.0×10⁷ U/100 g of superoxide dismutase (SOD).

7. The method of claim 1, wherein the aqueous G. tsugae extract comprises about 3.93% (w/w) water-soluble polysaccharide, about 1.96% (w/w) triterpene and about 4.31×10⁷ U/100 g of SOD.

8. The method of claim 1, wherein the ethanol extract of G. tsugae is obtained by heating a solution with G. tsugae and ethanol in a ratio of 1:10 to 30 at 70° C. to 85° C. for 80 to 120 hours

9. The method of claim 8, wherein the heating time is about 100 hours.

10. The method of claim 8, wherein the extract is obtained using 95% ethanol.

11. The method of claim 1, wherein the ethanol G. tsugae extract comprises at least 0.1% (w/w) water-soluble polysaccharide.

12. The method of claim 11, wherein the ethanol G. tsugae extract further comprises at least 10% (w/w) triterpene and 0.5×10⁸ U/100 g of SOD.

13. The method of claim 1, wherein the ethanol G. tsugae extract comprises about 0.22% (w/w) water-soluble polysaccharide.

14. The method of claim 13, wherein the ethanol G. tsugae extract further comprises about 21.7% (w/w) triterpene and about 1.32×10⁸ U/100 g of SOD.

15. The method of claim 1, wherein the hematological parameter is distribution of RBC volume, viscosity of whole blood, RBC aggregation index, erythrocyte electrophoresis index, erythrocyte rigidity index, internal viscosity of erythrocyte or oxygen transport efficiency.

16. A method for improving a brain dysfunction, comprising administering an effective amount of an ethanol or aqueous extract of G. tsugae obtained under high temperature to a subject.

17. The method of claim 1, wherein the brain dysfunction is a cognitive disorder.

18. The method of claim 1, wherein the brain dysfunction is memory impairment.

19. The method of claim 1, wherein the brain dysfunction is age-associated memory impairment, mild cognitive impairment, delirium, dementia, Alzheimer's disease, Parkinson's disease, Huntington's disease, mental retardation, cerebrovascular disease, affective disorders, psychotic disorders, Asperger's disorder, autism, neurotic disorders, attention deficit disorders, oppositional defiant disorder, conduct disorder, subdural hematoma, normal-pressure hydrocephalus, brain tumor, head trauma, or brain trauma.

20. The method of claim 1, wherein the brain dysfunction is depression or anxiety, psychosis, Down's syndrome, stroke, traumatic brain injury, Huntington's disease AIDS associated dementia, schizophrenia, or attention deficit disorder.
